(19) United States
(12) Patent Application Publication
(10) Pub. No.: US 2014/0320272 A1
(43) Pub. Date: Oct. 30, 2014

(54) Receptacle For Medical Instruments, And Method For Capturing And Transmitting Data From Medical Instruments

(71) Applicant: Rok Druzinic-Fiebach, Glienicke/Nordbahn (DE)

(72) Inventor: Rok Druzinic-Fiebach, Glienicke/Nordbahn (DE)

(21) Appl. No.: 14/329,155

(22) Filed: Jul. 11, 2014

Related U.S. Application Data
(63) Continuation of application No. PCT/EP2013/050458, filed on Jan. 11, 2013.

(30) Foreign Application Priority Data
Jan. 13, 2012 (DE) 102012000860.0

(51) Int. Cl. A61B 19/00

(52) U.S. Cl. CPC A61B 19/44; A61B 2019/448; A61B 2019/464
USPC 340/10.42

(57) ABSTRACT

A receptacle for medical instruments comprises a container for holding the medical instruments and comprises an apparatus for capturing and transmitting data from the medical instruments, which apparatus comprises a control unit, a reader and at least one first identification element attached to at least one medical instrument, wherein the control unit is connected to the reader and exchanges data with the reader, and performs a further step with said data, wherein the reader exchanges data with the at least one first identification element and passes said data to the control unit. The apparatus comprises a sensor unit which detects the medical instrument by a measurement performed thereon of a measurement quantity, and exchanges signals with the control unit, wherein the exchanged signals initiate the exchange of data between the reader and the at least one first identification element, wherein the reader transfers the exchanged data to the control unit.

### CROSS REFERENCES TO RELATED APPLICATIONS

This application is a continuation of international patent application PCT/EP2013/050458, filed on Jan. 11, 2013 designating the U.S., which international patent application has been published in German language and claims priority from German patent application DE 10 2012 000 860.0, filed on Jan. 13, 2012. The entire contents of these priority applications are incorporated herein by reference.

### BACKGROUND OF THE INVENTION

The present disclosure relates to a storage and/or transport receptacle for medical instruments, comprising a container for holding the medical instruments and comprising an apparatus for capturing and transmitting data from the medical instruments, which apparatus comprises a control unit, a reader and at least one first identification element attached to at least one medical instrument, wherein the control unit is connected to the reader and, actuated by signals, exchanges data with the reader, and processes, stores and/or passes to a second identification element attached to the container said data, wherein the reader exchanges data with the at least one first identification element and passes said data to the control unit.

US 2006/0244593 A1 shows a storage and/or transport receptacle for medical instruments, comprising a container for holding the medical instruments and comprising an apparatus for capturing and transmitting data from the medical instruments.

A storage and/or transport receptacle for medical instruments of the type mentioned in the introduction, comprising an apparatus for capturing and transmitting data from the medical instruments, can be used within the meaning of the present disclosure for holding, monitoring and tracking medical instruments in process operations within a hospital.

For this purpose, the storage and/or transport receptacles, referred to below also as hospital baskets, are identified in order to be able to track them in the processes operating within a hospital. The hospital baskets are typically identified by data matrix codes applied to the hospital baskets or by what are known as RFID transponders. These identification elements, which are typically attached to the hospital baskets, hence ensure good tracking of the hospital baskets by a corresponding reading facility within a hospital.

In this context and in the descriptions to follow, the abbreviation RFID stands for Radio Frequency Identification Device and denotes generally a device for wireless transmission of specific data.

The medical instruments located in such a hospital basket are also nor-mally equipped with such RFID transponders in order to ensure contactless capture of the medical instruments in the hospital basket. The medical instruments in the hospital basket can be captured by a reader mounted on or in the hospital basket, which reader then transfers the data determined from the medical instruments to the hospital reading facility.

In this process, the first identification elements, which are attached to the medical instruments, and the reader, which is mounted on or in the hospital basket, either exchange data continuously, or the data exchange is activated by a signal. In this case, the read process is either activated manually by, for instance, pressing a button on the hospital basket, or the read process is communicated by an external signal, which, for example, can be transferred to the reader of the hospital basket by the external hospital reading facility.

The aforementioned read processes have the disadvantage that they are either initiated manually and/or are very energy intensive, because the active read and transmit components of the hospital basket are actively exchanging data for a long time either with the medical instruments and/or with the external hospital reading facility.

In addition, it is disadvantageous that data is read from the medical instruments even when the biological, physical or chemical properties of these instruments have not changed, which can lead to an unnecessary load on the data infrastructure of a hospital.

### SUMMARY OF THE INVENTION

Hence it is an object to develop further a storage and/or transport receptacle for medical instruments of the type mentioned in the introduction, comprising an apparatus for capturing and transmitting data, such that data from the medical instruments and/or from the hospital basket can be transferred efficiently and according to need.

With regard to the storage and/or transport receptacle for medical instruments, which is mentioned in the introduction and comprises a container for holding the medical instruments and comprises an apparatus for capturing and transmitting data from the medical instruments, there is provided that the apparatus comprises a sensor unit which detects the at least one medical instrument by a measurement performed thereon of at least one measurement quantity, and exchanges signals with the control unit, wherein the exchanged signals initiate the exchange of data between the reader and the at least one first identification element, wherein the reader transfers the exchanged data to the control unit.

The apparatus disclosed in US 2006/0244593 A1, and the storage and/or transport receptacle for medical instruments described in this document, are based on the concept that the process of reading the transponders assigned to the medical instruments is activated by pressing a button and/or by a signal emitted from outside. According to this concept, activating the read process is based on an event that is induced manually either by pressing a button or by another signal.

In contrast, according to one aspect of the disclosure the apparatus according the concept of bringing about the read process manually is departed from in that the sensor unit integrated into the apparatus performs measurements on at least one medical instrument, and uses the measurements to determine measurement quantities specific to the medical instruments, which measurement quantities are processed by a control unit, and, after comparing the determined measurement quantities with threshold values stored in the control unit, if applicable causes activation of the reading process by the reader of the at least one first identification element attached to the at least one medical instrument.

The apparatus also comprises a sensor unit which, by virtue of a speci-fied measurement quantity of at least one medical instrument being reached, causes activation of the reading process by the reader of the at least one first identification element attached to the at least one medical instrument.

One aspect of the present disclosure is that a determination of the data specific to the medical instruments is only performed when necessary, i.e. if one of the measurement quantities of the instruments determined by the sensor unit changes sufficiently, has a direct impact on the time and effort associated with the data acquisition.

Since, according to one aspect, the data is only updated when necessary, said measure may have an effect on the entirety of the data traffic and on the reading facility needed for the read processes. In addition, one aspect disclosed herein has an effect on the energy resources provided by an energy storage device, because these resources only have to be used when necessary, i.e. when a measurement quantity determined by the sensor unit changes sufficiently relative to at least one threshold value.

A fault in the second identification element attached to the storage and/or transport receptacle, which can be caused by aging or damage for instance, can be rectified quickly by simply removing the second identification element from the container and replacing same without needing to open the storage and/or transport receptacle.

In a further refinement, the control unit comprises at least one memory element, at least one processing element, at least one transfer unit and at least one activation unit, wherein the activation unit is connected to the memory element, the processing element, the transfer unit and the reader for the purpose of activating processes for reading, processing and/or transferring data.

All the elements needed for determining, transferring, analyzing, processing and storing data may be combined in a central unit, which minimizes the complex-ity of the internal communication between the elements.

In a further refinement, the second identification element may comprise at least one data storage element and at least one transceiver element for exchanging data with an external transceiver unit.

A transceiver, a transceiver element, a transceiver unit, etc. are to be understood as entities comprising at least one of a transmitter and a receiver. The data relevant to the storage and/or transport receptacle or the contents thereof may be stored in the data storage element of the second identification element, and this data can be transferred, if applicable, to an external transceiver unit, which, according to some exemplary embodiments, may be in the form of an RFID facility.

According to some exemplary embodiments, the second identification element comprises at least one active transmitter, which, according to some exemplary embodiments, may be in the form of an active RFID transponder.

Using an active transmitter, which, in an exemplary embodiment, may be in the form of an active RFID transponder, may increase the range of the transmitter and hence the connectivity to the external transceiver units, which is beneficial for an interfer-ence-free data exchange.

In a further refinement, the reader comprises at least one transceiver element, which exchanges data wirelessly with the at least one first identification element.

The reader does not have to come into direct contact with the medical instruments in order to receive any necessary data. This form of contactless data transfer is particularly critical for the sterilization properties of the medical instruments.

In a further refinement the reader comprises at least one connection to the second identification element for the purpose of exchanging data.

The reader can exchange data directly with the second identification element without this data having to be processed and/or buffered first in the control unit.

It may be possible to store the determined data both in the second identification element and in the control unit, which reduces the risk of data loss.

In a further refinement, the sensor unit comprises at least one weight sensor, which detects the at least one measurement quantity in the form of the weight of at least one of the medical instruments.

According to some exemplary embodiments the weight sensor may be in the form of a piezoelectric element, wherein said piezoelectric element transfers electrical signals in the form of currents and voltages to the control unit.

A sensor in the form of a piezoelectric element can determine the existence of a medical instrument rather easily. Also, a separate power supply may not be needed for the piezoelectric element for detecting the weight of the medical instruments.

In a further refinement, the storage and/or transport receptacle comprises an energy storage device, wherein the sensor unit is connected to the energy storage device via the control unit.

The energy contained in the signals from the sensor unit may be routed to the energy storage device via the control unit as intermediary. This form of energy recovery is a further measure, in addition to the above-described activation of the signal-reading process performed by the reader, in order to extend the useful life of the storage and/or transport receptacle described herein, because the amount of energy still available in the energy storage device is the decisive factor limiting the useful life of said receptacle.

In a further refinement the second identification element may be connected to the energy storage device.

The reserves of energy in the energy storage element integrated in the second identification element may be conserved.

In a further refinement, an energy storage element integrated in the second identification element may be dispensed with by connecting the second identification element to the energy storage device, which significantly reduces the costs of said second identification element.

According to a further aspect, there is provided a method for capturing and transmitting data from medical instruments in a storage and/or transport receptacle of the aforementioned type, which method comprises the following steps:
a) placing at least one medical instrument onto a sensor unit,
b) the sensor unit and a control unit determining at least one measurement quantity of the at least one medical instrument,
c) the control unit processing the at least one measurement quantity,
d) the control unit activating a reader,
e) the reader capturing data from at least one first identification element of the at least one medical instrument engaging with the sensor unit,
f) transmitting the data determined by the reader to the control unit and/or to a second identification element,
g) the second identification element storing the data and/or making the data available in order to exchange data with at least one external transceiver unit.

The read process is actuated according to need on the basis of the above-mentioned method, and not activated by a manual signal that is actuated either by pressing a button or by another external signal.

In addition, the individual steps of the method can also be performed multiple times and/or the steps of the method can also be arranged in a different se-quence.

Moreover, further aspects may be obtained from the above description relating to the storage and/or transport receptacle.

In a further refinement, the method comprises the following steps in step b):
aa) determining a signal detected by the sensor unit and related to the at least one measurement quantity,
bb) converting the signal into the at least one measurement quantity,
cc) processing the at least one measurement quantity,
dd) transferring the energy contained in the signal to the energy storage device.

In addition to determining the measurement quantity, the energy contained in the form of currents and voltages in the sensor signal can be supplied to the energy storage device.

In a further refinement, the step cc) comprises the following steps:
comparing the at least one measurement quantity with at least one threshold value stored in a memory element,
an activation unit activating the reader when the at least one threshold value is exceeded.

By comparing the at least one measurement quantity with at least one threshold value stored in the memory element, external influences such as noise signals, vibrations and also other parasitic effects can be filtered out. This process is crucial, because in this process it is possible to distinguish an event that changes significantly the status of a medical instrument from a disturbance event.

According to some exemplary embodiments, as part of this comparison, a plurality of different biological, physical or chemical measurement quantities can also be compared with the respective threshold values stored in the memory element of the control unit, and also, in any combination of the determined measurement values, can cause activation of the reader.

In a further refinement, the sensor unit may comprise a pushbutton or switch, wherein by virtue of reaching a predetermined measurement quantity, the pushbutton or switch transfers electrical signals in the form of currents and voltages to the control unit.

The read process may be actuated directly by a predetermined measurement quantity being reached, which quantity is defined, for example, by the weight of at least one medical instrument, without comparing the measurement quantity with at least one threshold value stored in the memory element.

It shall be understood that the features mentioned above and those still to be explained below can be used not just in the particular combination stated but also in other combinations or in isolation without departing from the scope of the present disclosure.

Exemplary embodiments are described and explained below in greater detail with reference to some selected exemplary embodiments in conjunction with the enclosed drawings, in which:

It shall be understood that the features mentioned above and those still to be explained below can be used not just in the particular combination stated but also in other combinations or in isolation without departing from the scope of the present disclosure.

### BRIEF DESCRIPTION OF THE DRAWINGS

Exemplary embodiments are described and explained below in greater detail with reference to some selected exemplary embodiments in conjunction with the enclosed drawings, in which:

FIG. 1 shows a storage and/or transport receptacle and an external transceiver unit;

FIG. 2 shows a control unit of the storage and/or transport receptacle comprising the individual elements contained therein;

FIG. 3 shows a second identification element of the storage and/or transport receptacle comprising the individual elements integrated therein;

FIG. 4 shows a diagram of the elements of the storage and/or transport receptacle that are involved in the data-reading process;

FIG. 5 shows the elements relevant to processing, storing and transferring the data, and the interaction between said elements;

FIG. 6 shows the elements that are connected and can be connected to the energy storage device and a schematic diagram of the energy recovery; and

FIG. 7 uses a schematic flow diagram to show the method for capturing and transmitting data from medical instruments using the storage and/or transport receptacle.

### DETAILED DESCRIPTION OF THE INVENTION

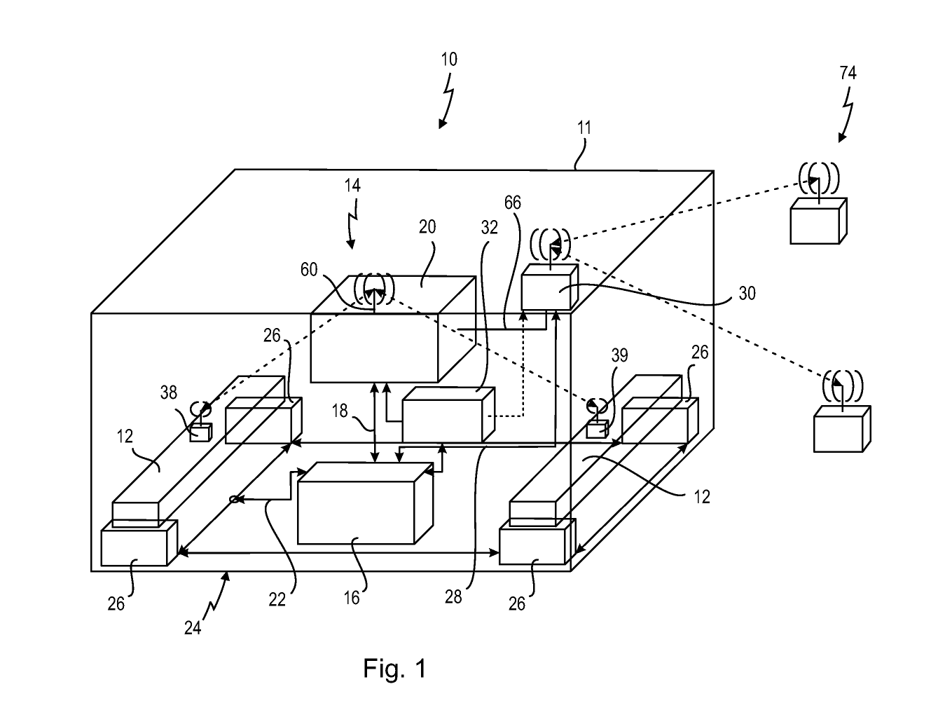

FIG. 1 shows schematically a storage and/or transport receptacle which is labeled with the general reference sign 10 and comprises a container 11 for holding the medical instruments 12. This storage and/or transport receptacle 10 additionally comprises an apparatus 14 for capturing and transmitting data from the medical instruments 12 located in the container 11. FIGS. 1 to 6 show further details of the storage and/or transport receptacle and of the apparatus 14 for capturing and transmitting data.

The apparatus 14 for capturing and transmitting data comprises at least one control unit 16, which is connected to a reader 20 via a first interface 18 and is represented schematically in FIG. 1 in the form of a double-ended arrow. In addition, the control unit 16 comprises a sensor interface 22, which is connected to a sensor unit 24 comprising at least one sensor 26 and is likewise denoted schematically by a double-ended arrow in FIG. 1. The control unit 16 further comprises a first external interface 28 to a second identification element 30, which interface is denoted schematically by a double-ended arrow.

The apparatus 14 for capturing and transmitting data additionally comprises an energy storage device 32, which supplies at least the control unit 16 and the reader 20 with power. According to some exemplary embodiments, the energy storage device 32 may be embodied by an electrochemical cell or by a capacitive memory element.

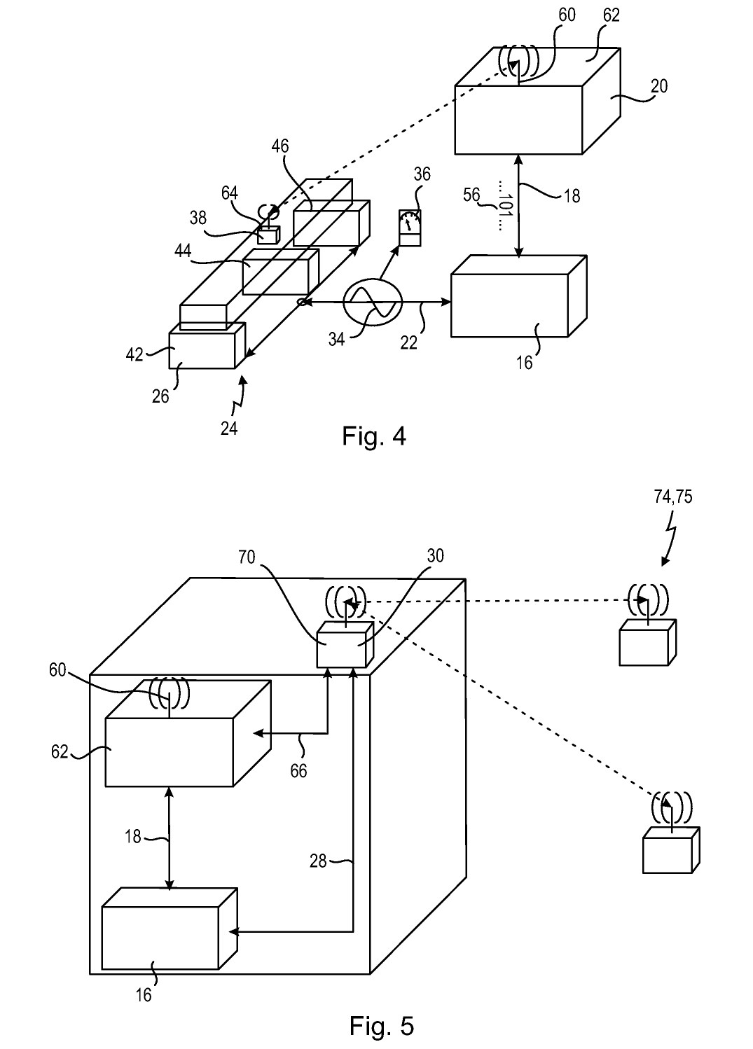

The control unit 16 receives from the sensor unit 24 via the sensor interface 22 signals 34 (FIG. 4), which are processed further in the control unit 16. The signals 34 are produced by measurements of at least one measurement quantity 36 (FIG. 4) on the medical instruments 12 that interact with the sensor unit 24.

The medical instruments 12 are themselves provided with identification elements 38, which can be connected wirelessly to the reader 20 for exchanging data.

Hence the signal 34 generated by a measurement by the sensor unit 24 on the medical instruments 12 can be used, inter alia, to establish a connection between the identification elements 38 and the reader 20 and to initiate thereby a data exchange between the reader 20 and the first identification element 38. The use of the signals 34 from the sensor unit 24 is not limited just to initiating the read process between the reader 20 and the at least one first identification element 38, but can be used in general to active or trigger the control unit 16 and/or the second identification element 30, which minimizes the power to be provided by the energy storage device 32 for supplying at least the control unit 16 and the reader 20.

It shall be understood that the at least one first identification element 38 is assigned individually to the medical instruments 12 in order to achieve a unique identification. Furthermore, additional medical instruments 12 can also be assigned additional identification elements 39.

According to some exemplary embodiments, the sensor unit 24 comprises at least one weight sensor 42 (FIG. 4), which detects the weight of the medical instruments 12. In addition, as shown in FIG. 4, the sensor unit 24 can have additional sensors 44 for determining additional physical, biological and/or chemical parameters of the medical instruments 12 and/or of the environment, such as e.g. the temperature, air humidity, pH value, magnetization and/or radioactivity. Again the signals 34 determined here can likewise be used, inter alia, for triggering the described device function, but also for other functions that are based on the measurement quantities 36 determined by the sensor unit 24.

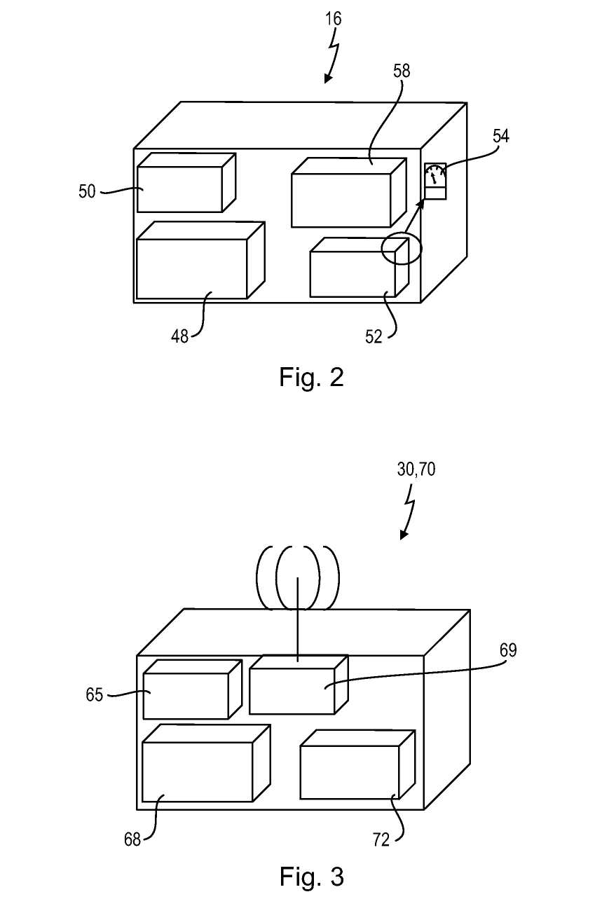

According to some exemplary embodiments, the weight sensor 42 is embodied by a piezoelectric element 46, which by virtue of the contact pressure of the medical instruments 12 passes signals 34 in the form of currents and voltages to a transfer unit 48 integrated in the control unit 16 (FIG. 2). As FIG. 2 likewise shows, the signals 34 are processed in the control unit 16 by the transfer unit 48, the processing element 50 and the memory element 52. Here, the signal 34 is converted into a measurement quantity 36 by the interaction of the transfer unit 48, the processing element 50 and the memory element 52. This measurement quantity 36 can then be compared with at least one reference value or threshold value 54 stored in the memory element 52.

In further embodiments, the sensor unit 24 can comprise a pushbutton or switch, which by reaching a measurement quantity 36 transfers signals 34 in the form of currents and voltages to the transfer unit 48 integrated in the control unit 16.

In a further embodiment, the pushbutton or switch, by virtue of the contact pressure imparted by placement of the medical instruments 12, can assume a predetermined position in which signals 34 in the form of currents and voltages are transferred to the control unit 16.

If in a weight measurement by the weight sensor 42 the threshold value 54 now equals exactly the weight of a medical instrument 12, it is possible to determine the existence or nonexistence of a medical instrument 12 by comparing the determined measurement quantity 36 and the threshold value 54. Hence the removal or addition of at least one medical instrument 12 can generate a trigger signal 56 by the interaction of the transfer unit 48, the processing element 50 and the memory element 52 (FIG. 4), which trigger signal is forwarded by an activation unit 58 at least to the reader 20 via the first interface 18.

The same obviously applies also to every physical, chemical and/or biological parameter for which a change thereto can be determined by a measurement on the medical instruments 12.

It shall be understood that both this and every other physical, biological and/or chemical measurement quantity 36 which is determined by the sensor unit 24 and processed in the control unit 16 can be stored either in the control unit 16 or in the integrated memory element 52, or can be transferred to the second identification element 30 via the transfer unit 48 and a first external interface 28.

The reader 20 can now perform by a transceiver element 60 the read process, activated by a trigger signal 56, as shown in FIG. 4, on the at least one first identification element 38.

According to some exemplary embodiments, the apparatus 14 is inac-tive, i.e. is in a standby mode, until the control unit 16 generates and forwards the trigger signal 56.

According to some exemplary embodiments, as shown in FIG. 5, the reader 20 may be designed as an active RFID reader or RFID-UHF reader 62 comprising active transceiver element 60, which exchanges data wirelessly with the at least one first identification element 38, which itself, according to some exemplary embodiments, may be designed as a passive RFID tag 64. In addition, the second identification element 30 comprises an energy storage element 65 for the supply of power.

It shall be understood that by communication with the reader 20 and the at least one first identification element 38, it is possible both to transmit data from the reader 20 to the first identification element 38 and to read data from the first identification element 38 by the reader 20. The data transfer with the reader 20 is thus ensured via a first interface 18, between the control unit 16 and the reader 20, and via a second external interface 66, between the reader and the second identification element 30.

The data determined from the at least one first identification element 38 can additionally be transferred to the data storage element 68 integrated in the second identification element 30 via the first external interface 28 (FIG. 1 and FIG. 5), which connects the control unit 16 to the second identification element 30, or via the second external interface 66, which connects the reader 20 to the second identification element 30.

The second identification element 30, which, according to some exemplary embodiments, may be embodied as an active RFID-UHF transponder 70 having active transmitter 69, sends/receives data by a second active transceiver element 72 to/from an external transceiver unit 74 located outside the apparatus 14, which transceiver unit, according to some exemplary embodiments, may be in the form of an active RFID-UHF facility 75.

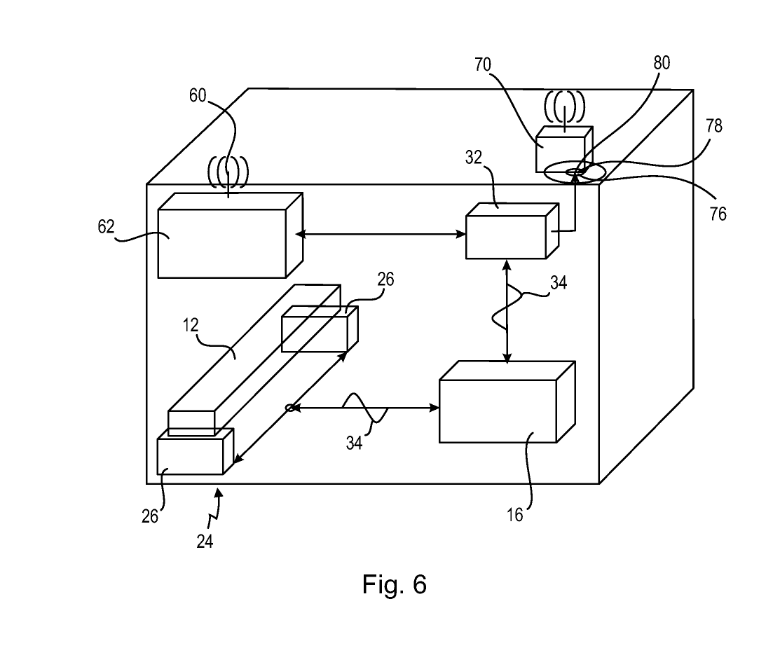

According to some exemplary embodiments, the signals 34 generated by the interaction of the medical instruments 12 with the sensor unit 24, as shown in FIG. 6, are used for energy recovery. Here, in a further exemplary embodiment, in which the sensor unit 24 comprises at least one piezoelectric element 46, the signals 34 generated by the weight measurement of the medical instrument 12 and composed of currents and voltages can be used for routing the energy contained therein via the control unit 16 to the energy storage device 32.

Now if the signal 34 corresponding to a measurement quantity 36 does not equal the threshold value 54 stored in the memory element 52 of the control unit 16 but is smaller than same, then simply all the energy stored in the signal 34 is transferred directly to the energy storage device 32 via the transfer unit 48 integrated in the control unit.

The signals 34 are here of a magnitude that corresponds to events during which the piezoelectric elements 46 detect a signal 34, for example caused by vibrations of the medical instruments or else other parasitic effects, without a medical instrument 12 being removed as a whole from the sensor unit 24 or from the piezoelectric element 46 directly. It is also necessary to distinguish between these two cases in order to filter out any noise signals that may interfere seriously with the operation of the process.

The same obviously applies also to every physical, chemical and/or biological parameter for which a change thereto can be determined by a measurement on the medical instruments.

In addition, all the energy contained in the signal 34 that is not needed for determining the measurement quantity 36 can be passed directly to the energy storage device 32 by the transfer unit 48.

The described transfer of the energy by the control unit 16 to the energy storage device 32 can also be applied similarly when the signals 34 corresponding to the measurement quantities 36 reach or exceed the threshold values 54 stored in the memory element 52.

This process is shown again for illustration purposes in FIG. 6. It can be seen here that the energy storage device 32 supplies at least the control unit 16 and the reader 20, which, according to some exemplary embodiments, may be an RFID reader 62, with power. As already described, the sensor unit 24, by virtue of the interaction effected by the medical instruments 12, can generate signals 34 in the form of currents and voltages, wherein the energy contained in the signals 34 can be routed via the control unit 16 to the energy storage device 32.

In a further exemplary embodiment, the second identification element 30 in the form of an active RFID transponder 70 is connected, for the purpose of the alterna-tive and/or additional supply of power, via an energy transfer interface 76 (FIG. 6) to the energy storage device 32 integrated in the apparatus 14. In a first exemplary embodiment, this interface is implemented as a capacitive and/or inductive interface 78 between the energy storage device 32 and the second identification element 30.

In a further exemplary embodiment, the energy transfer interface 76 is implemented by a metallic contact 80 between the energy storage device 32 and the second identification element 30.

The described form of the supply of power by a central energy storage device 32 may serve for the long service life and hence the cost-effectiveness of the described storage receptacle 10. Both the activation of the supply of power by signals 34 from the sensor unit 24 and also the energy recovery via the energy contained in said signals 34 and transferred by the control unit 16 to the energy storage device 32 can be handled centrally via an energy storage device 32 of the apparatus 14, which means that there is no need for an additional energy storage element 65 in the second identification element 30, or the energy reserves in the energy storage element 65 are conserved.

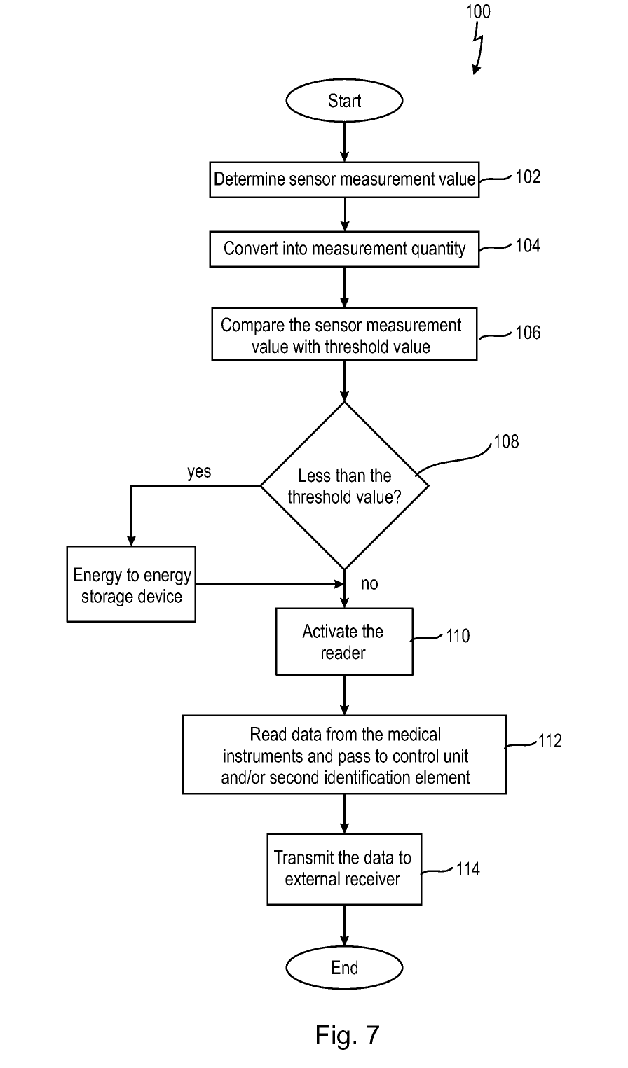

The method, denoted by the general reference number 100, for activating the read process by a reader 20, is reproduced again and summarized below with reference to a flow diagram shown in FIG. 7.

The method begins with the control unit 16 determining the sensor measurement value 102, wherein in the next step 104, the determined signal 34 at the sensor is converted by the control unit 16 into a measurement quantity 36. After comparing 106 the measurement quantity 36 with at least one threshold value 54 stored in the memory element 52 of the control unit 16, the processing element 50 and the memory element 52 interact to perform a differentiation step 108. If then the determined measurement quantity 36 lies below the threshold value 54 stored in the memory element 52, the energy contained in the signals 34 is passed directly to the energy storage device 32.

If the determined measurement quantity 36 reaches or exceeds the threshold value 54 stored in the memory element 52, by a further process step 110, the activation unit 58 will activate the read process communicated by the reader 20. The excess energy is supplied to the energy storage device 32.

In the next process step 112, the data that is stored in the at least one first identification element 38 is transferred via the reader 20 to the control unit 16 and/or to the second identification element 30.

In the next process step 114, the determined data is stored in the control unit 16 and/or in the second identification element 30, and/or is passed to an external receiver 74, which, according to some exemplary embodiments, may be in the form of an active RFID-UHF facility 75.

What is claimed is:

1. A receptacle for medical instruments, comprising a container for holding the medical instruments and comprising an apparatus for capturing and transmitting data of the medical instruments, which apparatus comprises a control unit, a reader and at least one first identification element attached to at least one medical instrument of the medical instruments, wherein the control unit is connected to the reader and, actuated by signals, exchanges data with the reader, and performs on said data at least one of a processing, storing, and passing to a second identification element attached to the container, wherein the reader exchanges data with the at least one first identification element and passes said data to the control unit, wherein the apparatus comprises a sensor unit which detects the at least one medical instrument by a measurement performed thereon of at least one measurement quantity, and exchanges signals with the control unit, wherein the exchanged signals initiate the exchange of data between the reader and the at least one first identification element, wherein the reader transfers the exchanged data to the control unit.

2. The receptacle of claim 1, wherein the control unit comprises at least one memory element, at least one processing element, at least one transfer unit and at least one activation unit, wherein the activation unit is connected to the memory element, the processing element, the transfer unit and the reader for the purpose of activating a process for at least one of reading, processing and transferring data.

3. The receptacle of claim 1, wherein the second identification element comprises at least one data storage element and at least one of a transmitter element and a receiver element for exchanging data with an at least one of an external transmitter element and an external receiver element.

4. The receptacle of claim 1, wherein the second identification element comprises at least one active transmitter.

5. The receptacle of claim 1, wherein the second identification element comprises at least one active RFID transponder.

6. The receptacle of claim 1, wherein the reader comprises at least one of a transmitter element and a receiver element, which exchanges data wirelessly with the at least one first identification element.

7. The receptacle of claim 1, wherein the reader comprises at least one connection to the second identification element for the purpose of exchanging data.

8. The receptacle of claim 1, wherein the sensor unit comprises at least one weight sensor, which detects the at least one measurement quantity as a weight of at least one of the medical instruments.

9. The receptacle of claim 1, wherein the weight sensor is a piezoelectric element, wherein said piezoelectric element transfers electrical signals in the form of currents and voltages to the control unit.

10. The receptacle of claim 1, wherein the apparatus comprises an energy storage device and wherein the sensor unit is connected to the energy storage device via the control unit.

11. The receptacle of claim 10, wherein the second identification element is connecta-ble to the energy storage device.

12. The receptacle of claim 1, wherein the sensor unit comprises a switch, wherein by virtue of reaching a predetermined measurement quantity, the switch transfers electrical signals in the form of currents and voltages to the control unit.

13. The receptacle of claim 1, wherein the sensor unit comprises a pushbutton, wherein by virtue of reaching a predetermined measurement quantity, the pushbutton transfers electrical signals in the form of currents and voltages to the control unit.

14. A method for capturing and transmitting data from at least one medical instrument in a receptacle, the method comprising the following steps:
a) placing at least one medical instrument onto a sensor unit,
b) the sensor unit and a control unit determining at least one measurement quantity of the at least one medical instrument,
c) the control unit processing the at least one measurement quantity,
d) the control unit activating a reader,
e) the reader capturing data from at least one first identification element of the at least one medical instrument engaging with the sensor unit,
f) transmitting the data determined by the reader to at least one of the control unit and a second identification element, and
g) the second identification element performing at least one of storing the data and making the data available in order to exchange the data with at least one external transceiver unit.

15. The method of claim 14, wherein step b) comprises the following steps:
aa) determining a signal detected by the sensor unit and related to the at least one measurement quantity,
bb) converting the signal into the at least one measurement quantity,
cc) processing the at least one measurement quantity, and
dd) transferring the energy contained in the signal to the energy storage device.

16. The method of claim 14, wherein step cc) comprises the following steps:
comparing the at least one measurement quantity with at least one threshold value stored in a memory element, and
an activation unit activating the reader when the at least one threshold value is exceeded.

17. The method of claim 14 wherein the receptacle comprises a container for holding the at least one medical instrument and comprising an apparatus for capturing and transmitting data of the at least one medical instrument, which apparatus comprises a control unit, a reader and at least one first identification element attached to the at least one medical instrument, wherein the control unit is connected to the reader and, actuated by signals, exchanges data with the reader, and performs on said data at least one of a processing, storing, and passing to a second identification element attached to the container, wherein the reader exchanges data with the at least one first identification element and passes said data to the control unit, wherein the apparatus comprises a sensor unit which detects the at least one medical instrument by a measurement performed thereon of at least one measurement quantity, and exchanges signals with the control unit, wherein the exchanged signals initiate the exchange of data between the reader and the at least one first identification element, wherein the reader transfers the exchanged data to the control unit.
